(19) United States
(12) Patent Application Publication
(10) Pub. No.: US 2008/0140174 A1
(43) Pub. Date: Jun. 12, 2008

(54) Method of increasing balloon flexibility in a balloon catheter

(75) Inventors: Randolf von Oepen, Los Altos Hills, CA (US); Lorcan Coffey, Tubingen (DE); Thomas Rieth, Hirrlingen (DE); Travis R. Yribarren, San Mateo, CA (US); Arik Zucker, Zurich (CH)

Correspondence Address: BEYER WEAVER LLP/Abbott Vascular Devices, P.O. BOX 70250, OAKLAND, CA, 94612-0250, US

(73) Assignee: Abbott Laboratories

(21) Appl. No.: 11/506,243

(22) Filed: Aug. 17, 2006

(51) Int. Cl. A61F 2/06

(52) U.S. Cl. 623/1.11; 604/523; 606/108

(57) ABSTRACT

A variety of methods are described that pre-condition a balloon used in a balloon catheter in order to increase its flexibility. Generally, the membrane that forms the balloon is crumpled in order to induce a large number of irregular crumples in the balloon. The crinkling of the balloon improves its flexibility and thus the flexibility of the catheter. Most typically, torsion and/or compression are applied to the balloon membrane in order to induce the crumpling. The described crumpling can be used to increase the flexibility of a balloon of a given strength in a variety of catheters, including angioplasty catheters and stent delivery catheters. In stent delivery catheters, the resulting crinkles also have the additional benefit of improving stent retention.

### BACKGROUND OF THE INVENTION

The present invention relates generally to techniques for increasing the flexibility of balloons used in catheters.

Catheters are used in a wide variety of medical procedures. Typically catheters are relatively long and flexible. Many times, (as for example in many vascular applications) a catheter is inserted into a relatively tortuous vessel. Many types of catheters carry a balloon that may be inflated as part of a medical procedure. For example, angioplasty catheters typically have balloons that are inflated in order to enlarge a narrowing (stenosis) in a blood vessel. Balloons are also typically used to deliver stents used in similar procedures.

As the distal end of a catheter travels through a vessel, it is frequently bent. Generally, balloons are considered quite flexible compared to other components that may be part of a particular catheter. However, increased flexibility is often considered a desirable characteristic for a balloon of a given strength. This is because balloons are typically carried near the distal end of the catheter and therefore less stiffness in the balloon translates to increased flexibility and better steerability at the distal end of the catheter.

Although existing devices work well there are continuing efforts to improve the flexibility of balloons used in various catheter applications.

### SUMMARY OF THE INVENTION

To achieve the foregoing and other objects of the invention, a variety of methods are described that pre-condition a balloon used in a balloon catheter in order to increase its flexibility. Generally, the membrane that forms the balloon is crumpled in order to induce a large number of irregular crumples in the balloon. The crinkling of the balloon improves its flexibility and thus the flexibility of the catheter, without significantly degrading the desirable properties (such as strength) of the balloon.

The crumpling can be accomplished in a variety of manners. Most typically, torsion and/or compression are applied to the balloon membrane in order to induce the desired crumpling.

The described crumpling can be used to increase the flexibility of a balloon of a given strength in a variety of catheters, including angioplasty catheters, stent delivery catheters and a wide variety of other balloon based catheters. In stent delivery catheters, the resulting crinkles also have the additional benefit of improving stent retention.

### BRIEF DESCRIPTION OF THE DRAWINGS

The invention, together with further objects and advantages thereof, may best be understood by reference to the following description taken in conjunction with the accompanying drawings in which:

FIG. 1 is a diagrammatic view of a simplified balloon catheter in accordance with one embodiment of the invention;

FIG. 2 is a diagrammatic view illustrating the crumpling of a balloon membrane using torsion in order to induce crinkling; and

FIG. 3 is a diagrammatic view illustrating the crumpling of a balloon membrane using compression in order to induce crinkling.

It is to be understood that, in the drawings, like reference numerals designate like structural elements. Also, it is understood that the depictions in the figures are diagrammatic and not to scale.

### DETAILED DESCRIPTION OF THE PREFERRED EMBODIMENTS

The present invention relates generally to techniques for increasing the flexibility of balloons used in catheters. More specifically, the invention contemplates crumpling the membrane that is used as a balloon in a balloon catheter in order to induce the irregular ridges and valleys associated with crinkling.

Balloons used in most balloon catheters are formed from relatively smooth membranes. Typically, when a balloon is mounted on a catheter it is folded around the catheter so that it maintains a relatively low profile. Thus, the balloon has a number of fold lines, but otherwise, the pleats of the folded balloon tend to be relatively smooth. We have determined that the flexibility of the balloon, and therefore the catheter, can be improved somewhat by inducing irregular crinkles into the balloon.

In order to induce crinkles into the balloon, the membrane that forms the balloon may be crumpled prior to its installation on the catheter. As will be described in more detail below, the crumpling can be accomplished using a variety of different techniques, but for the purpose of understanding the invention, it is perhaps easiest to envision a process that is akin to crumpling a piece of paper. The membrane that forms the balloon is a thin sheet that is somewhat similar to a sheet of paper. When the sheet (or the balloon membrane) is crumpled, a large number of irregular ridges, valleys, divots, fold lines and the like are formed. These types of irregular structures may be referred to as “crimples”, “crumples” or “rumples”, and are generally distinguished from the more regular folds and crease lines that are formed by pleating or more neatly folding a sheet or membrane.

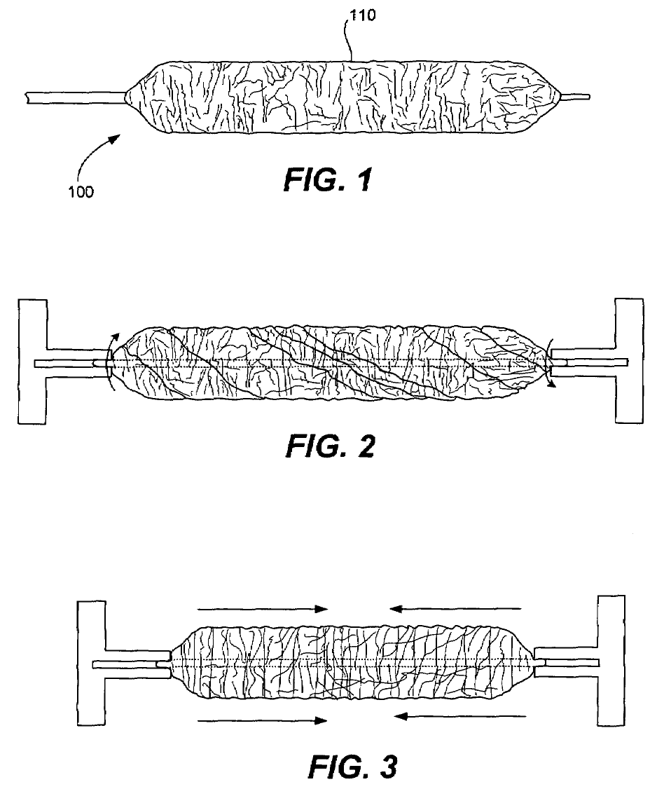

The balloon is preferably crumpled before it is mounted on the catheter. Thus, from a process standpoint, the balloon is crumpled and uncrumpled before it is mounted on the catheter. Generally, the balloon may be mounted on the catheter using any standard or suitable balloon mounting technique. A representative balloon catheter 100 incorporating a crumpled balloon 110 in accordance with the present invention is diagrammatically illustrated in FIG. 1.

The strength of the balloon does not appear to be compromised in any significant amount by the crumpling, while at the same time, the flexibility of the balloon increases.

As pointed out above, the crumpling can be accomplished using a wide variety of techniques. By way of example, one suitable approach is to mount the balloon on a mandrel, grip both ends of the mandrel and twist one end of the balloon to induce the crimping. Such an arrangement is diagrammatically illustrated in FIG. 2. Alternatively, both ends of the balloon may be twisted in opposing directions. In another approach, the balloon may be mounted on a mandrel and compressed to induce the crimping. Such an arrangement is diagrammatically illustrated in FIG. 3. Alternatively both compression and twisting may be used at the same time. In still other approaches, the twisting and/or compression may be performed without the use of a mandrel.

The best effects are believed to occur if the entire balloon is crumpled, however, depending on the technique used to induce the crumples, some portions of the membrane (e.g., the ends) may not be crumpled due to the constraints of the handling equipment.

Although only a few embodiments of the invention have been described in detail, it should be appreciated that the invention may be implemented in many other forms without departing from the spirit or scope of the invention. For example, FIG. 1 illustrates a simplified catheter arrangement. It should be appreciated that in many applications, the catheters will have more sophisticated designs that are specific for their intended tasks. For example, many catheters employ multi-lumen tubular members. Additionally, there are a wide variety of different mechanisms that can be used to induce the crumpling and the magnitude of the crumpling may vary significantly. Therefore, the present embodiments are to be considered as illustrative and not restrictive and the invention is not to be limited to the details given herein, but may be modified within the scope and equivalents of the appended claims.

What is claimed is:

1. A method of fabricating a balloon catheter, the method comprising:
crumpling a flexible membrane to induce irregular crumples in the flexible membrane;
uncrumpling the flexible membrane, whereby the uncrumpled flexible membrane had a number of irregular crumples and ridges; and
installing the uncrumpled flexible membrane on a catheter having an inflation port, wherein the flexible membrane is directly or indirectly sealed to the catheter at a location that overlies the inflation port so that the flexible membrane forms a balloon that may be inflated by injecting a fluid through the inflation port.

2. A method as recited in claim 1 wherein the crumpling is accomplished by applying torsion to the flexible membrane.

3. A method as recited in claim 2 further comprising positioning the flexible membrane on a mandrel and wherein the torsion is applied to the flexible membrane by twisting at least one end of the flexible membrane.

4. A method as recited in claim 1 wherein the flexible membrane has a tubular geometry and the crumpling is accomplished by applying a combination of compression and torsion to the flexible membrane.

5. A method as recited in claim 1 wherein the crumpling is accomplished by applying compression to the flexible membrane.

6. A method as recited in claim 1 further comprising the step of installing a stent over the balloon in a collapsed state, whereby the stent may be deployed by actuating the balloon.

7. A balloon catheter comprising:
an elongated tubular member suitable for insertion into a body vessel, the elongated tubular member having a port and an inflation lumen, the port being positioned near a distal end of the elongated tubular member and in fluid communication with the inflation lumen; and
an expandable balloon carried by the elongated tubular member, wherein the port opens into an interior space of the balloon such that the balloon may be inflated by supplying fluid to the balloon through the inflation lumen, wherein the expandable balloon is folded in a collapsed position and has a multiplicity of irregular crumples formed by precrumpling the balloon to improve the flexibility of the balloon.

8. A stent delivery catheter comprising:
a balloon catheter as recited in claim 7;
a stent secured to the catheter in a collapsed state over the balloon, whereby the stent may be deployed by inflating the balloon.
